(19) United States
(12) Patent Application Publication
(10) Pub. No.: US 2026/0216150 A1
(43) Pub. Date: Jul. 30, 2026

(54) VDAC MODULATORS FOR BLISTERING SKIN CONDITIONS

(71) Applicants: THE MEDICAL RESEARCH, INFRASTRUCTURE, AND HEALTH SERVICES FUND OF THE TEL AVIV MEDICAL CENTER, Tel Aviv (IL); THE NATIONAL INSTITUTE FOR BIOTECHNOLOGY IN THE NEGEV LTD., Beer Sheva (IL)

(72) Inventors: Eli SPRECHER, Givatayim (IL); Ofer SARIG, Ramat Yishai (IL); Sari ASSAF, Mi'ilya (IL); Varda SHOSHAN-BARMATZ, Omer (IL)

(73) Assignees: THE MEDICAL RESEARCH, INFRASTRUCTURE, AND HEALTH SERVICES FUND OF THE TEL AVIV MEDICAL CENTER, Tel Aviv (IL); THE NATIONAL INSTITUTE FOR BIOTECHNOLOGY IN THE NEGEV LTD., Beer Sheva (IL)

(21) Appl. No.: 19/109,624

(22) PCT Filed: Sep. 7, 2023
(86) PCT No.: PCT/IL2023/050963

§ 371 Date: Mar. 7, 2025

(51) Int. Cl. A61K 31/438; A61K 45/06; A61P 17/00

(52) U.S. Cl. CPC A61K 31/438; A61K 45/06; A61P 17/00

(57) ABSTRACT

The invention provides compositions and methods for treating a group of rare autoimmune blistering skin conditions resulting from defective epidermal and mucosal cell adhesion such as pemphigus and pemphigoid.

### TECHNOLOGICAL FIELD

The invention generally pertains to the field of therapies for autoimmune skin disorders, and specifically for rare blistering skin conditions resulting from defective epidermal and mucosal cell adhesion such as pemphigus and pemphigoid.

### BACKGROUND

Cell-cell adhesions are necessary for structural integrity and barrier formation of the epidermis. Cell adhesion is provided by specialized cell-cell junctions, primarily adherens junctions (AJ), desmosomes (patch-like intercellular adhering junctions), and tight junctions. In addition to forming physical connections between epidermal cells, these junctions organize and regulate cytoskeletal elements and modulate signaling pathways to regulate tissue development, structure, and physiology and communication between epidermal and subepidermal layers. Defective epidermal cell adhesion contributes to the pathogenesis of some rare autoimmune skin conditions. Two examples are pemphigus and pemphigoid, both result from abnormal production of autoantibodies against certain components of cell-cell adhesion machinery.

Pemphigus and pemphigoid are rare blistering autoimmune diseases manifested in blisters and sores on the skin or mucous membranes, including the mouth and genitalia. Although they differ in spread and severity of symptoms and site of action of the autoimmune antibodies, the hallmark of both conditions is significant loss of epidermal and subepidermal cell adhesion that accompanies blister and bullous formation.

Pemphigus affects the outer epidermal layer of the skin and is usually associated with the appearance of characteristic lesions and blisters that are easily ruptured. Pemphigoid affects the lower skin layers, between the epidermis and dermis, and is recognized by “tense” blisters or hives or eczema without blisters. Both conditions can have blistering lesions on mucous membranes such as mucosa of the mouth and eyes. Specific examples are Pemphigus Vulgaris (PV), the most common type of pemphigus, and Mucous Membrane Pemphigoid (MMP). Most types of pemphigus are chronic and can be fatal if remain untreated. Bullous Pemphigoid, which is the more predominant in elderly patients, is usually less severe and rarely involves mucosa, and is treatable.

The underlying mechanism of both these conditions is an “overreaction” of the immune system and production of autoantibodies that attack the healthy epidermal and subepidermal tissues and mucous membranes which eventually results in the formation of blisters, accumulation of fluid within or between epidermal and mucosa layers, ultimately leading to epidermal and/or subepidermal cell detachment. In pemphigus, the autoantibodies are directed against components of cell-cell adhesion machinery in the upper epidermis, such as Desmoglein 1 and 3 adhesion proteins (dsg1 and dsg3). In pemphigoid, the autoantibodies are directed against cell-cell adhesion components in the basal epidermal layer and mucosa, such as membrane associated and transmembrane glycoproteins BP antigens 1 and 2 (BPA-1 and BPA-2, also BPAG1 and BPAG2).

Currently, there is no cure for pemphigus or pemphigoid and the existing treatments are predominantly symptomatic. In addition, the severity and progression of these conditions are not uniform and the response to therapies substantially varies among individuals. For the most part, pemphigus is easier to control. The mainstay treatments are corticosteroids such as prednisone and other anti-inflammatory medications, the general disadvantage of which is in suppressing the normal immune function. Steroids are usually administered systemically, per os or by injection. Topical treatments are predominantly given to reduce pain and to treat infections. Rituximab, a monoclonal antibody used for other autoimmune disorders and certain types of cancers, is now considered a first-line therapy for pemphigus. It was recently approved by the FDA for this indication.

An experimental approach to the treatment of various types of autoimmune disorders was suggested in WO 2020/110111, using specific modulators of voltage-dependent anion channel (VDAC) such as piperazine- and/or piperidine-derivatives. This publication, however, did not provide specific solutions for autoimmune skin conditions and not specifically for disorders of defective epidermal and mucosal cell adhesion.

### GENERAL DESCRIPTION

The invention stems from surprising findings by the inventors, showing that overexpression of the ST18 gene, a C2H2C-type zinc finger transcription factor, was associated with up-regulation of VDACs in human keratinocytes in vitro. ST18 is a known tumor suppressor gene partaking in the regulation of apoptosis. More recent genetic studies pointed to a potential link between S18 and the pathogenesis of PV.

A study in Egyptian and Jewish populations showed that a single nucleotide polymorphism (SNP) in the ST18 gene is associated with an increased risk for PV under dominant inheritance model. Another study showed that overexpression of ST18 accelerates PV IgG-induced cell-cell detachment and further contributes to PV IgG-induced blister formation.

The mammalian mitochondria express three voltage-dependent-anion channel (VDAC) isoforms, VDAC1, VDAC2, VDAC3, of which VDAC1 is the most abundant and therefore the most studied. VDAC1 mediates a metabolic crosstalk between mitochondria and the rest of the cell in facilitating the transport of metabolites such as pyruvate, malate, succinate, nucleotides and NADH into the mitochondria, and an inverse transport of newly formed molecules such as ATP and hemes out of mitochondria. VDACs, in general, are further involved in the transport of cholesterol, ions and reactive oxygen species (ROS) thereby regulating the mitochondrial and cytosolic redox states. VDAC1, specifically, has been implicated in triggering of apoptosis via mediation of release of a series of apoptogenic proteins, such as cytochrome C, apoptosis inducing factor (AIF) and the mitochondrial protein SMAC from the mitochondria to the cytosol. WO 2020/110111 reported on the involvement of VDACs in certain immunological responses, and further suggested that inhibition of the VDAC activity can positively affect certain autoimmune conditions exemplified in an animal model of lupus.

However, the involvement VDACs in cell adhesion and their potential effect on conditions stemming from deficiencies of cell-cell interaction is less well characterized, and specifically autoimmune skin conditions resulting from loss of epidermal and subepidermal cell adhesion such as pemphigus and pemphigoid.

To explore potential applicability of VDACs to pemphigus and other disorders stemming from the deficiencies of skin cell adhesion, the inventors developed several important experimental tools. To that end, the inventors established several operative models in vitro mimicking PV. One was a two-dimensional monoclonal HaCat cell model stably transfected with ST18 as a putative marker of PV condition. Another was the AK23-induced model in primary keratinocytes, using anti Desmoglein 3 (dsg3) to mimic the PV autoantibodies. This latter was further enhanced by introducing TNFα, yet another PV effector known to be elevated in PV serum and associated with negative PV outcomes. The inventors further developed a Dispase-based keratinocyte Dissociation Assay (DDA) in vitro to evaluate the effect of different stimuli on the loss of keratinocyte cell-adhesion.

Using the HaCat cell model, the inventors could show that the overexpression of ST18 was tightly associated with significant up-regulation of all three VDACs, VDAC1, 2 and 3, and down-regulation of Bel-2 which is a known anti-apoptotic agent. The link between S18 and VDACs was further supported in a study in vivo of PV patients carrying the rs17315309 allele which is known to promote ST18 overexpression. Analysis of VDAC1 in skin biopsies of these patients showed that VDAC1 was significantly elevated in the epidermal tissue of PV patients carrying the risk allele compared to patients with the wild type allele and healthy controls. Using DDA in primary keratinocytes exposed to PV serum and control, the inventor could show that the cells exposed to PV serum displayed significantly increased cell detachment compared to the cells exposed to the normal serum. (EXAMPLE 1)

Having these tools, the inventors hypothesized that an inference with VDAC pro-apoptotic activity can rescue the defects of epidermal cell adhesion that are characteristic of PV. Previous studies suggested that oligomerization of VDACs is a prerequisite for mitochondria-mediated apoptosis. A group of compounds, piperazine and piperidine derivatives, were reported in WO 2020/110111 as effective inhibitors of VDAC oligomerization, including a specific piperidine derivative (1-(naphthalen-2-ylmethyl)-4-(phenylamino) piperidine-4-carbonyl)glycine referred to as VBIT-12. The inventors have applied VBIT-12 in the in vitro models mimicking the PV condition.

Initial evidence for potential applicability VBIT-12 to disorders of epidermal cell adhesion dysfunction was provided in experiments in the AK23-induced/TNFα model in primary human keratinocytes overexpressing ST18. This system per se overexpresses VDACs, as would expected from an authentic PV model, while application of VBIT-12 in this system significantly attenuated the induction of VDAC by AK23 Ab. Furthermore, looking into specific molecular cues triggered by VBIT-12 in this system, it became apparent that VBIT-12 interferes with the VDACs activity along the p53-triggered pro-apoptotic pathway and thereby leads to diminution of apoptosis. This notion was supported by several findings: 1st, the finding of the down-regulating effect of VBIT-12 on the activity of pro-apoptotic enzymes, caspases 3/7 (EXAMPLE 2.1); 2nd-its down-regulating effect on keratinocytes cell death and apoptosis (EXAMPLE 2.2); 3rd-its up-regulating effect on the expression of Bcl-2 protein, an anti-apoptotic marker (EXAMPLE 2.3); and 4th-its down-regulating effect on the p53 transcriptional activity (EXAMPLE 2.4).

Moreover, using DDA in the AK23-induced model of acantholysis, it was possible to show that the anti-apoptotic effect of VBIT-12 could rescue the loss of cell adhesion, a hallmark of PV, which was evident by significant reduction acantholysis with increased concentrations of VBIT-12 and reversal of acantholysis caused by a small Bel-2 inhibitor, ABT-199, (EXAMPLE 3)

Taken together, these findings reinforce the notion that PV and other related skin disorders can be treated or relieved through inhibition of VDAC-mediated apoptosis. Moreover, they point to an alternative mechanism of action of VDAC inhibitors in the context of skin and their direct effect on epidermal and/or subepidermal cells adhesion and acantholysis. In other words, they show the distinctive features of VDAC action in the epidermal and subepidermal tissues and the consequences of an interference with this action for epidermal and subepidermal cell adhesion, and further point to distinctive molecular cues or markers that can help to monitor these processes. Ultimately, they provide proof of concept for the applicability of VBIT-12 and other VDAC inhibitors to skin disorders associated with defects of epidermal and/or subepidermal cell adhesion.

Current studies are focusing on corroboration and expansion of these findings in the mouse model of PV, using VBIT-12 as a model drug, alone and in combination with other therapeutic agents that are relevant to the treatment of PV and attenuation of its progression. Additional studies explore other methods of interference with VDAC apoptotic activity, such as by silencing with specific siRNA, and using other inhibitors of VDAC oligomerization.

### BRIEF DESCRIPTION OF THE DRAWINGS

To better understand the subject matter and to exemplify how it may be carried out in practice, embodiments will now be described by way of non-limiting examples with reference to the following drawings.

FIGS. 1A-1B illustrate the relationship between the overexpression of ST18 (mimicking PV) and the overexpression of VDAC in a human keratinocytes cell model. HaCat cells were stably transfected with ST18 or empty vector (EV) and subjected to sequence analysis of total RNA. Data was validated by qPCR (*p<0.05, *** p<0.001 by 2-tailed t test). Figures show that the overexpression of ST18 was accompanied by significant up-regulation of all three VDACs, VDAC1, 2 and 3, and by a down-regulation of Bel-2, which is a known anti-apoptotic agent (1A and 1B, respectively).

FIGS. 2A-2C illustrate the manifestation of epidermal overexpression of VDAC1 in PV patients carrying the rs17315309 allele that was related to overexpression of ST18. Immunohistochemistry staining for VDAC1 was performed in skin biopsies of healthy controls (n=5) and PV patients with the wild type allele (n=5) and the rs17315309 risk allele (n=3) (2A, 2B and 2C, respectively; scale bar=100 μm). Figures show significant up-regulation of VDAC1 expression in PV patients with the risk allele.

FIG. 3 illustrates the manifestation of loss of epidermal cell adhesion (a hallmark of PV) as revealed by DDA assay in vitro. A keratinocyte cell monolayer was exposed to serum obtained from a PV patient (containing PV IgG) and a control and further subjected to DDA. Figure shows significant cell detachment in the cells exposed to the PV serum.

FIGS. 4A-4B illustrate the effect of VBIT-12 on apoptosis as revealed in the AK23-induced/TNFα model that was tested for Caspase 3/7 activity (a pro-apoptotic marker). Primary human keratinocytes were transfected with ST18 expression vector (ST18) or with empty vector (EV) and exposed to AK23 (10 ug/mL)/NC recombinant human TNF-α (20 ng/mL) and VBIT-12/DMSO, Caspase 3/7 activity was measured by Caspase 3/7 Glo activity assay. Results represent means+SE from 3 independent experiments (*p<0.05 by 2-tailed t test). Figures show that while cells overexpressing ST18 had elevated caspase 3/7 activity, VBIT-12 exerted significant down-regulation of the Caspase 3/7 activity (4A), quantitated as 50% reduction activity relative to PV mimicking conditions (S18) and a negligible effect in normal control (EV) (4B).

FIG. 5 illustrates the effect of VBIT-12 on apoptosis as revealed by quantitative TUNEL assay in the same system. Cells were immunostained for cell death using the TUNEL and DAPI staining, and the number of TUNEL positive cells was quantified. Results represent means+SE from 3 independent experiments (*p<0.05 by 2-tailed t test). Figure shows that VBIT-12 had significant effect on attenuation of apoptosis, with a reduction of about 50% in cell death relative to PV mimicking conditions (S18).

FIGS. 6A-6D illustrate the effect of VBIT-12 on the expression of Bel-2 (an anti-apoptotic protein) in the same system. The Bcl-2 expression was measured and quantified using immunofluorescence staining (6A-6B) and immunoblotting (6C-6D). Results represent means+SE from 3 independent experiments (*p<0.05 by 2-tailed t test). Figures show that while the S18 overexpressing cells had significantly down-regulated levels the Bcl-2 protein, VBIT-12 reversed this down-regulation almost up to the normal levels (EV controls) by both quantitative analyses.

FIG. 7 illustrates the effect of VBIT-12 on the transcription activity of p53 (a pro-apoptotic agent and a transcription factor), using a functional assay for p53 transcriptional activity by a luciferase reporter with a p53 binding motif that was introduced into the same system. Results represent means+SE from 3 independent experiments (*p<0.05 by 2-tailed t test). Figure shows that while the p53 transcriptional activity was significantly up-regulated in the PV mimicking system (AK23), VBIT-12 reduced the p53 activity almost to the level of normal control (NC).

FIGS. 8A-8B illustrate the effect of VBIT-12 in the model of AK23-induced acantholysis (mimicking PV) as revealed by DDA in vitro. Primary human epidermal keratinocytes were exposed to AK23 (3.75 μg/mL)/NC and VBIT-12/DMSO. Epidermal sheets were released with dispase and subjected to mechanical stress (cell fragments were counted by two independent evaluators). Results represent means+SE from 3 independent experiments (*p<0.05, by 2-tailed t test). Figures show a significant reduction in the number of cell fragments with increased concentrations of VBIT-12 (8A), suggesting specificity and dose repose. Representative images of cell sheet fragmentation in the different groups are also shown (8B).

FIGS. 9A-9C illustrate the effect of VBIT-12 on the disinhibition of Bel-2 in the same system, using a specific Bel-2 inhibitor, Venetoclax (ABT-199). Cells were exposed to AK23/NC and VBIT12/DMSO and further exposed to ABT-199 and tested by DDA. Results represent means+SE from 3 independent experiments (*p<0.05, by 2-tailed t test). Figures show that while the normal controls (NC) displayed insignificant acantholysis (9A), the PV mimicking system displayed increased acantholysis, per se (AK23/DMSO) and more so in the presence of the Bcl-2 inhibitor (DMSO/ABT-199), and that VBIT-12 reversed the effect of the Bel-2 inhibitor (VBIT-12/ABT-199) (9B). Representative images of cell sheet fragmentation in the different groups are also shown (9C).

FIG. 10 illustrates a putative mechanism of action of VDAC inhibitors in the context of PV, with the example of the VBIT-12 inhibitor of VDAC oligomerization. Figure shows possible interference of VBIT-12 with the p53-triggered apoptotic pathway, involving Bcl-2 (negative effector) and caspases 3/7 (positive effectors) in regulation of apoptosis. VBIT-12 acts upstream to caspases 3/7 in mitigating the effects of caspases and ST18 (positive effector), thus ultimately leading to rescue of epidermal cell adhesion (adapted from Philomena George, Int Curr Pharm Res Vol 3, Issue 2).

### DETALED DESCRIPTION OF SPECIFIC EMBODIMENTS

In the broadest sense, the present invention provides a treatment solution for specific types of autoimmune skin conditions stemming from deficiencies or loss of epidermal and/or subepidermal cells adhesion. Cell-cell adhesion is governed by complex crosstalk between multiple junctional systems that are responsible for mechano-sensational, electrochemical and molecular transduction of signals between adhesion proteins. Numerous studies revealed noncanonical roles of cell adhesion proteins and their involvement in many aspects of tissue physiology, including growth control, differentiation, and inflammation. It is becoming increasingly apparent that junctional crosstalk, interdependencies and compensation are at the root of tissue integrity.

Desmosomal control of signaling is one of the important determinants of normal skin integrity, while chronic desmosomal disfunction can lead physiological remodeling of epidermal and subepidermal tissues and the emergence of bullous diseases. Known examples are pemphigus or a pemphigoid where loss of cell adhesion is induced by autoantibodies against desmosomal adhesion proteins, dag1 and dag3.

Thus, the term “skin disorder” implies herein a group of disorders the common feature of which is loss or a persistent deficiency of epidermal and/or subepidermal cells adhesion.

In some embodiments the skin disorder can result from loss or a deficiency in the functionality of the epidermal and/or subepidermal junctional complexes.

In some embodiments the skin disorder can result from loss or a deficiency in the functionality of adherens junctions.

In some embodiments the skin disorder can result from loss or a deficiency in the functionality of desmosomes.

In some embodiments the skin disorder can result from loss or a deficiency in the functionality of tight junctions.

In some embodiments the skin disorder can be an autoimmune skin disorder resulting from abnormal production of autoantibodies against one of these junctional complexes.

The consequences of these deficiencies can have various clinical manifestations involving internal and external epidermal and subepidermal linings.

In some embodiments the skin disorder can be a bullous disease. The term “bullous disease” implies herein a group of conditions generally referred as dermatoses, characterized by blisters and bullae in the skin and mucous membranes that can affect various parts of the body, including skin, hair, and oral, genital and ocular mucosa.

For treating these types of conditions, the invention provides an innovative therapeutic approach using modulators of voltage dependent anion channels, VDACs. The term “VDAC” or “VDACs” implies herein mitochondrial proteins localized at the outer mitochondrial membrane. It further implies the three known VDACs isoforms, VDAC1, 2 or 3, and further VDACs oligomeric, dimeric, trimeric and tetrameric forms. It predominantly implies the human VDACs, including VDAC1 encoded by the respective gene on human chromosome 5, VDAC2-on human chromosome 10 and VDAC3-on human chromosome 8. This term further implies the entire range of VDACs associated activities, including their primary activities in bilateral transport of metabolites, ions and other molecules between mitochondria and cytosol, and their broader involvement in apoptosis, immunological responses and cell adhesion, and many other cellular processes. It further relies on the notion that VDACs oligomerization and subsequent formation of a large channel is a prerequisite for VDACs mediated activities.

As has been demonstrated in this application, in the context of skin VDACs act as mediators of apoptosis and acantholysis, manifested in loss or deficiency of epidermal and/or subepidermal cell-cell interaction and cell adhesion and premature cell death.

The term “apoptosis” implies herein the sum of intracellular events that underlie programmed cell death, including disintegration of cellular DNA, proteins and a series of enzymatic reaction triggering thereof. It further implies various forms of apoptotic cell death, including anoikis which is a type of apoptosis characterized by inadequate cell-matrix interaction and apoptolysis which refers to epithelial cell adhesion. This latter overlaps with the term “acantholysis” which refers to loss of unity between epidermal cells due to the breakdown of intercellular bridges. Both apoptolysis and acantholysis imply loss of epidermal and/or subepidermal cell-cell interaction and cell adhesion.

The term “modulator” implies herein any interference with VDACs structure and/or function that can lead to alteration of one or more of VDACs activities.

In some embodiments the modulator can be an inhibitor of one or more of VDACs activities.

In some embodiments the modulator can be an inhibitor of VDACs pro-apoptotic activity. Alterations of VDACs pro-apoptotic activity can be measured by various known methods, one of them is TUNEL staining and quantification of DNA fragments which are exemplified in this application.

In some embodiments the modulator can be an inhibitor of VDACs induced acantholysis. Alterations VDACs-mediated acantholysis can be assessed by various methods, including DDA in vitro which was exemplified in this application.

In some embodiments the modulator can be an inhibitor of VDAC1 activity.

In some embodiments the modulator can be an inhibitor of VDAC1 pro-apoptotic activity and/or VDAC1 induced acantholysis.

In some embodiments the modulator can be a protein, a peptide or an antibody.

In some embodiments the modulator can be a small molecule drug or agent, or a small RNA molecule interfering with VDACs expression and/or activity.

In some embodiments the modulator can be an inhibitor of VDACs oligomerization.

In some embodiments the modulator can be a piperazine-derivative or a piperidine-derivative, which are known inhibitors of VDAC1 oligomerization, or an enantiomer, a diastereomer, a mixture or a salt thereof. Certain candidates from this groups that proved to be effective VDAC1 inhibitors were described in WO 2020/110111

In some embodiments the inhibitor of VDAC1 oligomerization can be a piperidine derivative (1-(naphthalen-2-ylmethyl)-4-(phenylamino) piperidine-4-carbonyl)glycine, referred to herein as VBIT-12, or an enantiomer, a diastereomer, or a salt thereof. General structure of these compound is given below:

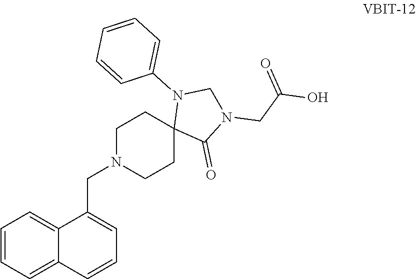 [SMILES: O=C(O)CN1CN(c2ccccc2)C2(CCN(Cc3cccc4ccccc34)CC2)C1=O]

Thus, in the broadest sense, the present invention can be articulated in terms of compositions, methods and uses for slowing the progression or treating a skin disorder associated with a defect of epidermal and/or subepidermal cells adhesion, the distinctive feature of which is that the composition comprises an effective amount of at least one modulator of at least one voltage-dependent anion channel isoform VDAC1, 2 or 3.

In some embodiments the composition can be formulated as a topical composition or topical formulation that is administered directly onto the skin, i.e., non-systemically.

In other embodiments the composition can be formulated as an oral composition that is administered via an oral or sublingual routes to induce systemic effects.

In some embodiments the skin disorders that are treated by the compositions and methods of the invention can further comprise a defect of cells adhesion in oral, genital and/or ocular mucosa.

In some embodiments the skin disorders can comprise a bullous disease, which is a group of conditions. Differential diagnosis of conditions from this group is made in routine by a dermatologist or other medical practitioner specializing in the diagnosis and treatment of skin disorders.

In some embodiments the bullous disease can be related to loss or a defect of desmosomal integrity and control, such as in pemphigus and pemphigoid disorders.

In some embodiments the bullous disease can be a type of pemphigus or a pemphigoid. The terms “pemphigus” and “pemphigoid” imply herein a group of conditions that are usually distinguished by clinical presentation, lesion biopsy, histopathological evaluation and identification of autoantibodies. Some examples of specific types of pemphigus or a pemphigoid are provided below.

Pemphigus Vulgaris (PV), the most common of these conditions, involves blisters that are soft and fragile and may form at the mouth first and then spread to the skin and even the genitals. PV does not cause permanent scarring unless there is an infection associated with the sore.

Pemphigus Foliaceus (PF), a less severe type, may have blisters on the scalp and face first and then spread to the chest and back. Blisters do not occur in the mouth. Blisters are superficial and form crusts.

Pemphigus Vegetans, this type results in thicker sores, mainly in the groin and under the arms.

IgA Pemphigus, caused by the IgA antibody binding to epidermal cell proteins. It may resemble pemphigus foliaceus or may appear as small pustules.

Paraneoplastic Pemphigus (PNP), associated with certain forms of cancer, with blisters forming inside the mouth and may affect the lungs, leading to a fatal outcome. Sores of the mouth, lips and esophagus are almost always present and skin lesions of different types occur.

Mucous Membrane Pemphigoid (MMP) may affect the eyes, mouth and throat.

A clinical form called ocular cicatricial pemphigoid (OCP) can result in blindness if it involves the eyes and respiratory compromise if it involves the deeper parts of the throat.

Bullous Pemphigoid (BP) is limited to the skin, with blisters presenting predominantly on the abdomen, groin, back, arms and legs.

Gestational Pemphigoid (GP), characterized by a blistering rash starting around the navel and spreading to the entire body, typically in the second trimester of pregnancy.

Epidermolysis Bullosa Acquisita, involving a blistering rash on the skin without involvement of mucosal surfaces. Blisters are usually smaller than in pemphigoid.

In some embodiments the bullous disease can be Dermatitis herpetiformis commonly known as a celiac disease.

In some embodiments the bullous disease can be a Skin fragility disorder (also Ectodermal dysplasia), which is a group of conditions characterized by skin fragility and blistering, palmoplantar keratoderma, abnormal hair growth, nail dystrophy, and occasionally defective sweating.

In some embodiments the bullous disease can be selected from Staphylococcal scalded skin syndrome, Toxic epidermal necrolysis, Severe cellulitis.

In some embodiments the bullous disease can be related to loss or a defect of functionality of adherens junctions, such as in various inflammatory conditions of skin and hair.

In some embodiments the bullous disease can be related to loss or a defect of functionality of tight junctions such as in atopic dermatitis and psoriasis.

An important distinguishing feature of the present compositions and methods is in providing evaluation of disease progression and effectiveness of treatment. The terms “effective amount” and “therapeutically effective amount”, which are used herein in conjunction with VDACs modulators, imply an amount, a concentration or a dose of the modulator that are sufficient for inducing a measurable reduction of one or more clinical symptoms, or histological, molecular or biochemical markers of the disease.

In some embodiments the therapeutic effect of the compositions and methods of the invention can be recognized by a reduction of apoptosis in the epidermal and/or subepidermal cells, which can be detected by respective assays in the skin biopsies of the treated patients. Such technologies have been presently exemplified.

In some embodiments the therapeutic effect of the compositions and methods of the invention can be recognized by a reduction of acantholysis in the epidermal and/or subepidermal cells, which can be detected by respective assays in the skin biopsies of the treated patients. Such technologies have been presently exemplified.

In some embodiments the therapeutic effect of the compositions and methods of the invention on the skin disorder can be recognized by one of more markers of disease progression that have been identified in this application, i.e.:
a. a reduction of activity of at least one of caspase 3, 7 and/or 9 in the epidermal and/or subepidermal cells in the skin biopsies of the treated patients,
b. a reduction of transcription activity of p53 in the epidermal and/or subepidermal cells in the skin biopsies of the treated patients,
c. an increase in the expression of Bcl-2 gene or protein in the epidermal and/or subepidermal cells in the skin biopsies of the treated patients.

Relevant assays for detecting these markers in epidermal derived cells have been presently exemplified. More generally, methods of detecting changes in enzyme activity, and specifically caspase 3, 7 and/or 9, are well known in the art, as well as methods for detecting and quantifying expression levels of specific genes or proteins. Methods for detecting changes in transcription activity of specific factors are also well established, by means of dependent genes or gene promoters that can be internal or external to the system.

In some embodiments the compositions and methods of the invention can be a part of combination therapies using one or more additional therapeutic agents, comprised either in the compositions or administered in succession independently thereof. Examples of such agents are corticosteroids such as prednisone, Rituximab which is now considered a first-line therapy for pemphigus, and other anti-inflammatory and immunosuppressive drugs such as mycophenolate mofetil, azathioprine, methotrexate, or cyclophosphamide. Among more recent candidates are antibody targeting the FcRN receptor and certain types of tyrosine kinase inhibitors.

Ultimately, the invention can be articulated in terms of use the above-described compositions in the manufacture of a medicament for slowing the progression or treating a skin disorder associated with a defect of epidermal and/or subepidermal cells adhesion.

### EXAMPLES

### Example 1: Development of PV In-Vitro Model in Human Keratinocytes

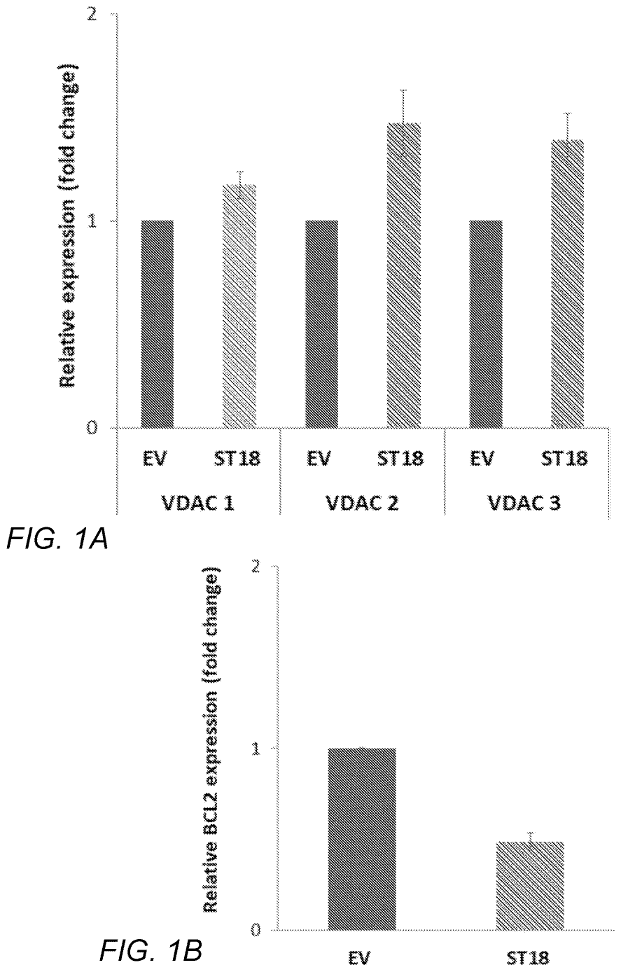

The inventors developed a PV model in-vitro using two-dimensional monoclonal HaCat cell line (keratinocytes) stably transfected with ST18 or empty vector (EV), where they could study the ST18-modulating effect of potential therapeutic agents. Initial characterization of this system using sequencing analysis of total RNA suggested that the overexpression of ST18 (PV mimicking conditions) was tightly associated with significant up-regulation of all three VDACs, VDAC1, 2 and 3, and down-regulation of Bcl-2, which is a known anti-apoptotic agent. The results are shown in FIGS. 1A-1B.

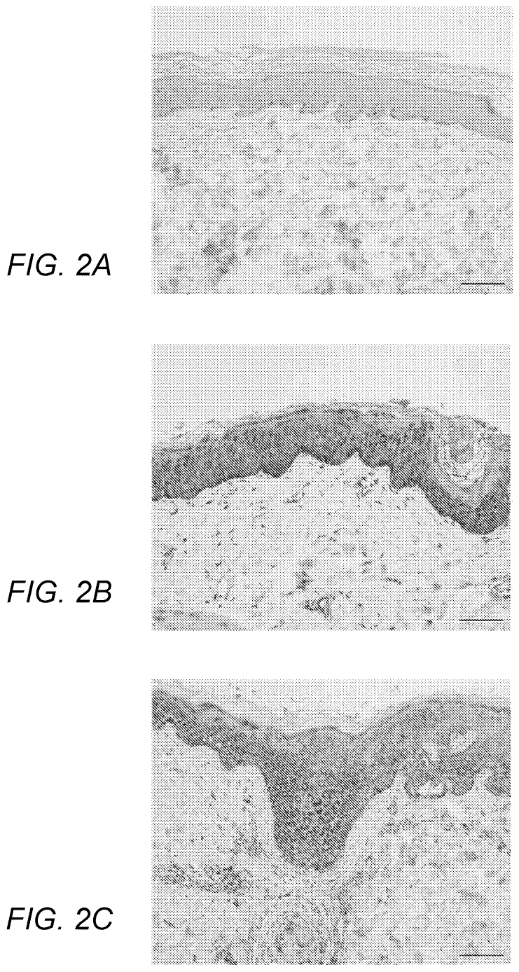

The link between S18 and VDACs was further supported in a study ex-vivo of PV patients carrying the rs 17315309 allele which is known to promote ST18 overexpression. Using immunohistochemistry staining for VDAC1 in skin biopsies of these patients vs. PV patients with wild types alleles and healthy controls, the inventors could show that VDAC1 was significantly elevated in the epidermal tissue of PV patients carrying the rs 17315309 risk allele. The results are shown in FIGS. 2A-2C.

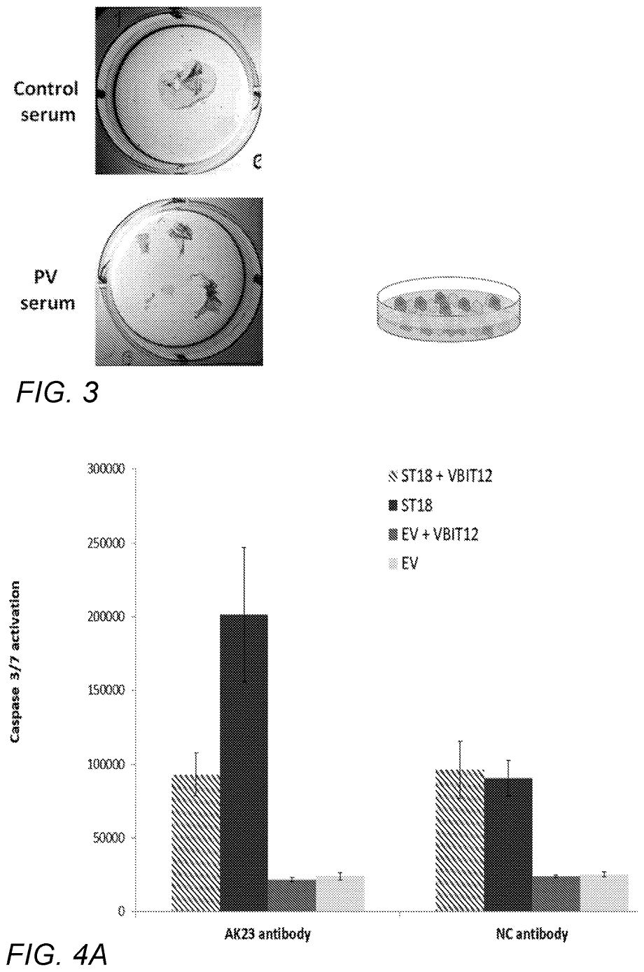

The inventors have further developed a Dispase-based keratinocyte Dissociation Assay (DDA) in vitro. DDA permits to evaluate the effect of different stimuli on the loss of keratinocyte cell-cell interaction. The cells are exposed to a specific stimulus and then to dispase-based detachment, mechanical stress (e.g., pipetting or tilting) and quantification of the resulting fragments. The inventors could show that after exposing a monolayer of primary epidermal keratinocytes to PV serum containing PV IgG, the cells exhibited increased dissociation from the culture dish compared to the cells exposed to the normal control serum. The results are shown in FIG. 3.

### Example 2: The Effect of VBIT-12 on AK23 Induced Apoptosis

Having these tools, the inventors hypothesized that an inference with VDAC pro-apoptotic activity can rescue the defects of epidermal cell adhesion that are characteristics of PV. One of the options to interfere with the VDACs activity is via inhibiting VDACs oligomerization. The mechanisms by which VDACs form functional protein-conducting channels is not well understood, but there is accumulated evidence suggesting that VDACs can exist in oligomer, dimer and trimer forms, and that oligomeric VDACs and specifically VDAC1 may be involved in mitochondria-mediated apoptosis. An interference with VDAC oligomerization can be achieved by various approaches, including the use of piperazine- and/or piperidine-derivatives. Certain inhibitors of VDAC oligomerization were developed in WO 2020/110111, including a specific piperidine derivative with the chemical formula (1-(naphthalen-2-ylmethyl)-4-(phenylamino) piperidine-4-carbonyl)glycine, referred to therein as VBIT-12.

For mimicking the effect of PV, the inventors used anti-desmoglein 3 antibody, AK233 antibody (AK23 Ab). Desmoglein 3 (dsg3) is a 130 kDa polypeptide, and like the pemphigus foliaceus antigen (dsg1) is a member of the desmoglein subfamily. Autoantibodies against dsg1 and dsg3 are characteristic of PV. When applied to primary human keratinocytes overexpressing ST18, AK23 Ab induces the expression of VDAC, that can be visualized by use of immunofluorescence staining, as would be expected from an authentic PV model. Using VBIT-12 in this system, the inventors could show that the VBIT-12 inhibitor significantly attenuated the induction of VDAC by AK23 Ab (immunofluorescence images not shown), thus providing initial evidence for applicability of this VDAC inhibitor for disorders of epidermal cell adhesion dysfunction.

### 2.1 the Effect of VBIT-12 on AK23 Induced Caspase Activation

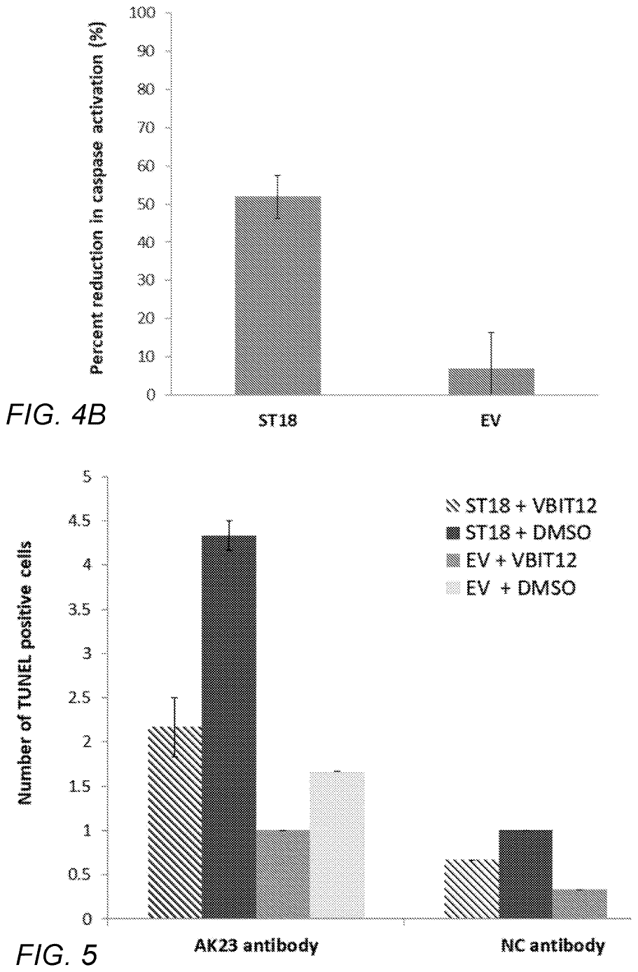

The AK23-induced model of PV was further investigated regarding the effect of VBIT-12 on various indices of apoptosis, the initial choice was caspases 3/7 which are known markers of apoptosis. The experimental system was further enhanced by incorporation of tumor necrosis factor alpha (TNFα). The notion of relevance of TNFα to the PV pathogenesis stems from several findings, including the clinical data on elevated levels of TNFα the serum of PV patients and their association with negative PV outcomes, and further, the finding that the ST18 transcription factor acts on the TNFα promoter. Thus, TNFα was incorporated into the AK23-induced model of PV. Introducing VBIT-12 into this model, the inventors could show that while cells overexpressing ST18 (PV mimicking conditions) display elevated caspase 3/7 activity, VBIT-12 exerted significant down-regulation of the caspase 3/7 activity, with 50% reduction relative to PV mimicking conditions (S18) and a negligible effect in normal control (EV). The results are shown in FIGS. 4A-4B.

### 2.2 the Effect of VBIT-12 on AK23-Induced Cell Death

The effect of VBIT-12 on apoptosis was further was further corroborated by studies using quantitative TUNEL assay (detecting DNA fragmentation associated with cell death). While the AK23-induced/TNFα system per se displayed as significant number of TUNEL positive cells, the system exposed to VBIT-12 had a significantly reduced number of TUNEL positive cells, which is indicative a reduced apoptosis. Upon quantification of the visual data (immunofluorescence images not shown), the inventors could show that VBIT-12 had significant effect on attenuation of apoptosis, with a reduction of about 50% of cell death relative to PV mimicking conditions (S18) that matched the findings on caspase 3/7 in the same system. The results are shown in FIG. 5.

### 2.3 The Effect of VBIT-12 on AK23 Bcl-2

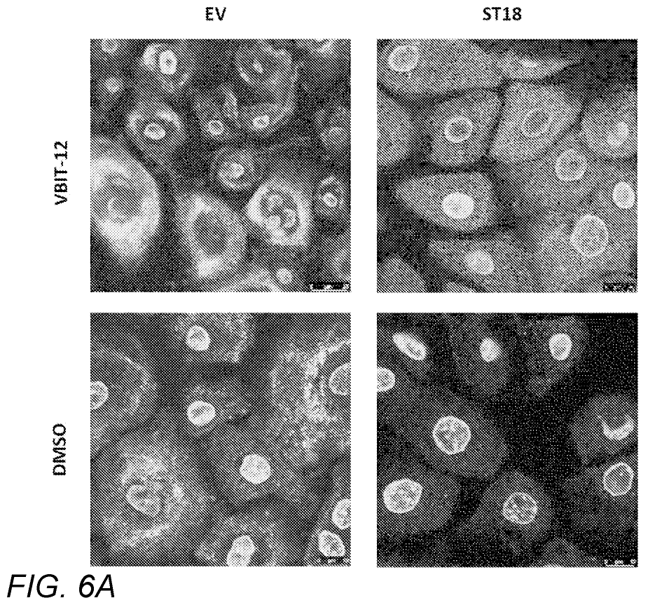

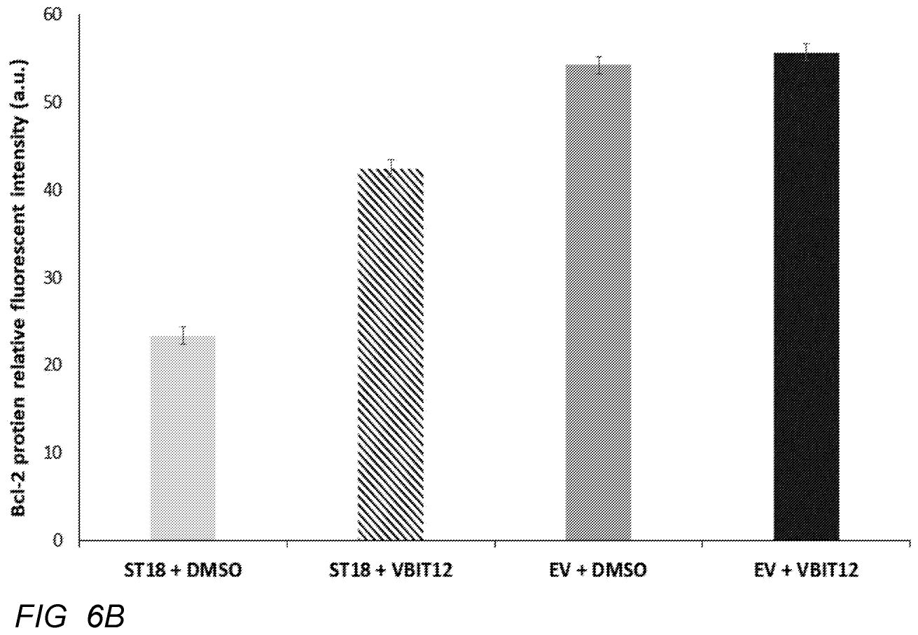

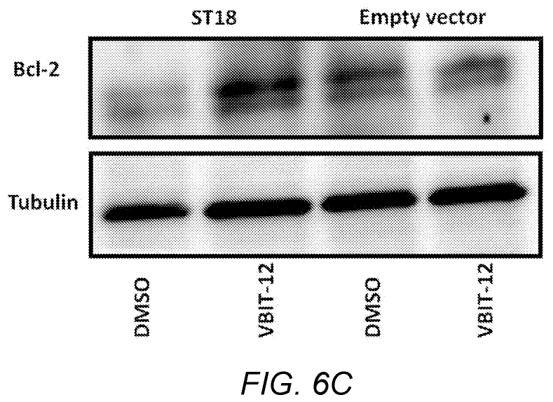

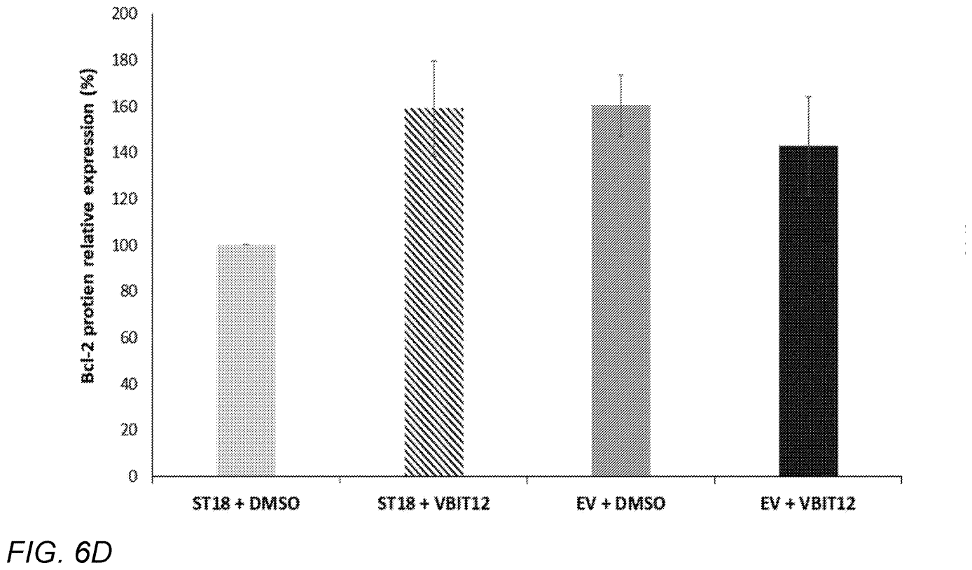

As has been noted, Bel-2 is a known anti-apoptotic agent. The inventors have further found that it is significantly down-regulated in the $18 overexpressing model of human keratinocytes, as opposed to the up-regulated VDACs in the same system (see EXAMPLE 1). Using the the AK23-induced/TNFα system, the inventors could show that that while the Bcl-2 protein was down-regulated in the S18 overexpressing cells, VBIT-12 reversed this down-regulation almost up to the normal levels (EV controls). The results are shown in FIGS. 6A-6D.

### 2.4 The Effect of VBIT-12 on p53 Transcription Activity

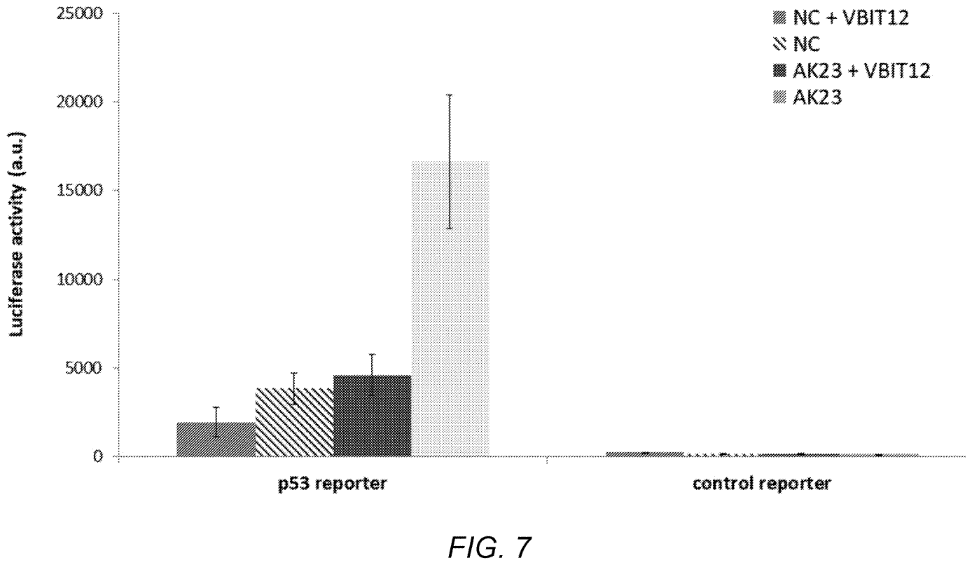

Another interesting marker of apoptosis is p53, p53 is a transcriptional regulator which is known to act as a pro-apoptotic agent. In this respect its effect on apoptosis is analogous to caspases 3/7 and opposed to Bcl-2. The effect of VBIT-12 on p53 was assessed by a functional assay for p53 transcriptional activity using a luciferase reporter with a p53 binding motif that was introduced simultaneously together with all other relevant agents into the AK23-induced/TNFα system. Using this approach, the inventors could show that while the p53 transcriptional activity was significantly up-regulated in the PV mimicking system (AK23), VBIT-12 reduced the p53 activity almost to the level of normal control (NC). The results are shown in FIG. 7. These results were further consistent with the down-regulating effect of VBIT-12 on the activity of caspases 3/7 and its up-regulating effect on the expression of Bcl-2 and its overall down-regulating effect on the AK23-induced cell death that were revealed in previous experiments (see EXAMPLES 2.1-2.3).

### Example 3: The Effect of VBIT-12 on AK23 Induced Acantholysis

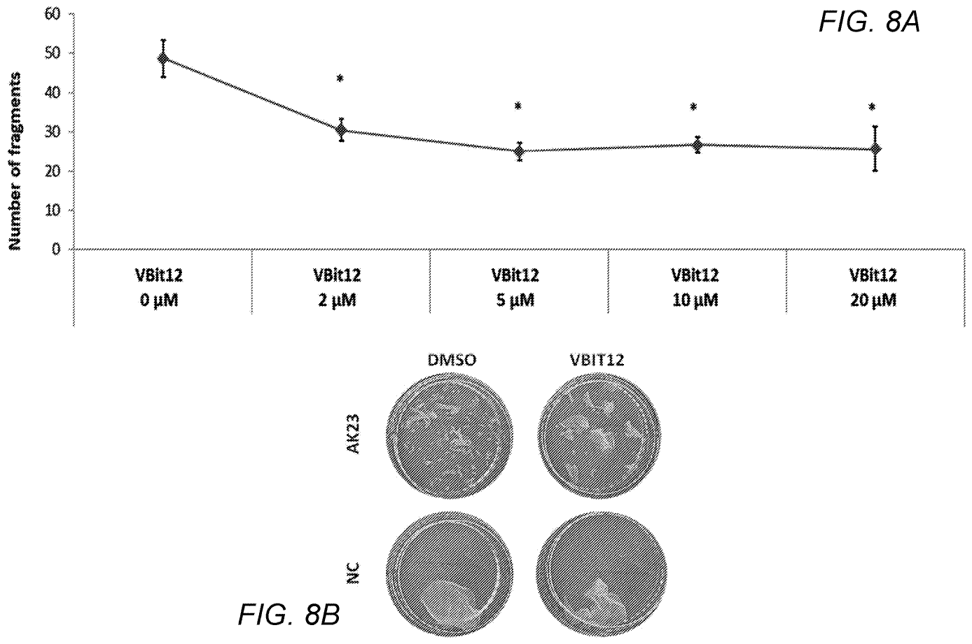

The inventors have further investigated the effect of VBIT-12 in the model of AK23-induced acantholysis. Epidermal cell detachment (acantholysis) or deficient epidermal cell adhesion is a hallmark of PV. Mimicking the PV condition in vitro with AK23-induced primary keratinocytes and employing the previously developed DDA for evaluating the propensity to acantholysis, the inventors could show significant reduction acantholysis with gradually increased concentrations of VBIT-12, suggesting specificity and reliable dose repose. The results are shown in FIGS. 8A-8B.

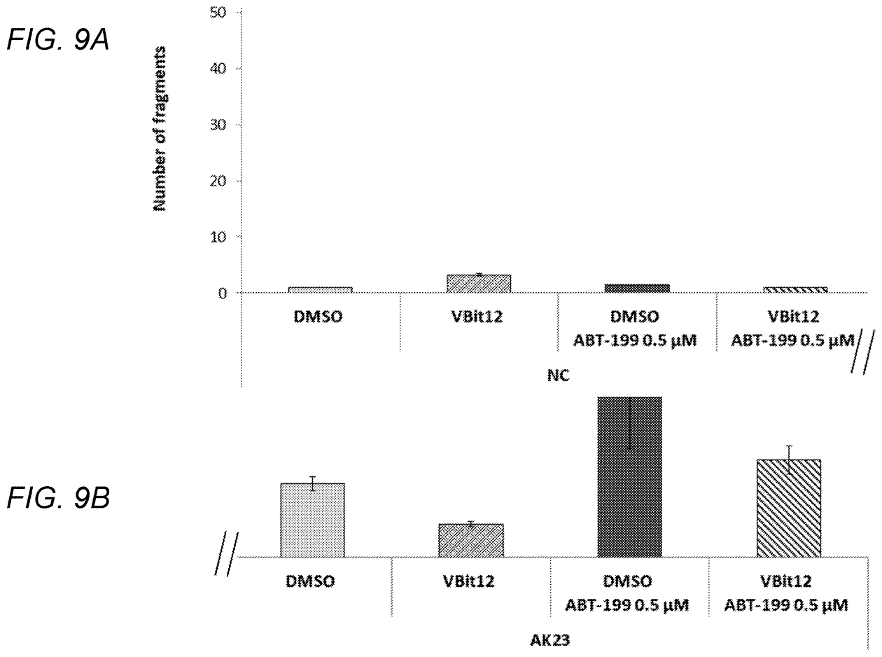

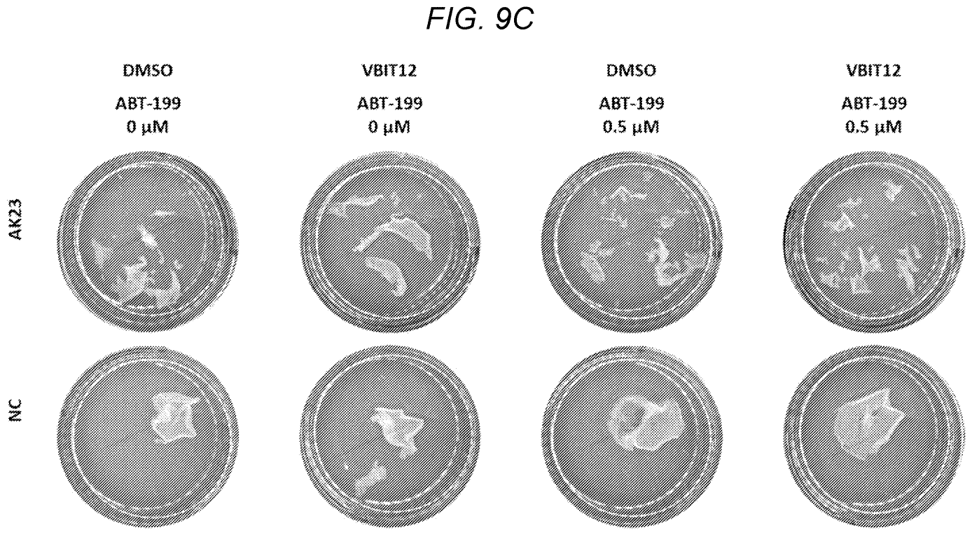

Further support for consistency of these findings was provided by evaluation of Bel-2 in the same experimental system, per se and in the presence of a specific Bel-2 inhibitor, Venetoclax (ABT-199). The inventors have found that while the normal controls displayed lower acantholysis relative to the PV mimicking system, introduction of the Bel-2 inhibitor into the system increased the degree of acantholysis, when VBIT12 could reverse the effect of the Bel-2 inhibitor. The results are shown in FIGS. 9A-9C.

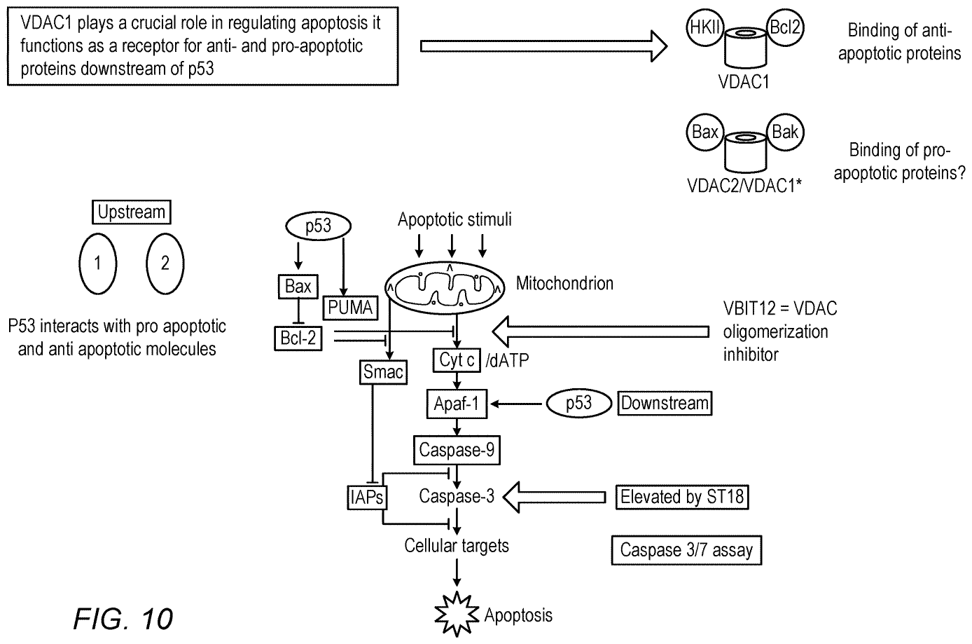

Ultimately, the inventors have proposed a putative mechanism of action of VDAC inhibitors in the context of PV, with the example of the VBIT-12 inhibitor of VDAC oligomerization. The inventors suggested that VBIT-12 acts along the p53-triggered apoptotic pathway that further involves Bcl-2 as an up-stream negative effector and caspases 3/7 as down-dream positive effectors in regulation of apoptosis. VBIT-12 acts upstream to caspases 3/7 in mitigating the effects of caspases and ST18, another positive effector of apoptosis, thus ultimately leading to rescue of epidermal cell adhesion. A graphic illustration of the proposed model is provided in FIG. 10.

1.-41. (canceled)

42. A method for slowing the progression or treating a skin disorder associated with a defect of epidermal and/or subepidermal cells adhesion in a human subject, the method comprises topical or oral administering to said subject a therapeutically effective amount at least one modulator of at least one voltage-dependent anion channel isoform VDAC1, 2 or 3.

43. The method of claim 42, wherein the skin disorder further comprises a defect of cells adhesion in oral, genital and/or ocular mucosa.

44. The method of claim 42, wherein the skin disorder comprises a bullous disease.

45. The method of claim 44, wherein the bullous disease is selected from Pemphigus vulgaris, Pemphigus foliaceus, Bullous pemphigoid, Dermatitis herpetiformis, Skin fragility disorders.

46. The method of claim 44, wherein the bullous disease is selected from Staphylococcal scalded skin syndrome, Toxic epidermal necrolysis, Severe cellulitis.

47. The method of claim 44, wherein the bullous disease is a type of pemphigus or a pemphigoid.

48. The method of claim 42, wherein the at least one VDAC modulator is an inhibitor of VDAC oligomerization.

49. The method of claim 42, wherein the at least one VDAC modulator is a modulator of the VDAC isoform 1 (VDAC1).

50. The method of claim 49, wherein the modulator of VDAC1 is an inhibitor of VDAC1 oligomerization.

51. The method of claim 50, wherein the inhibitor of VDAC1 oligomerization is a piperazine-derivative or a piperidine-derivative, or an enantiomer, a diastereomer, a mixture or a salt thereof.

52. The method of claim 51, wherein the inhibitor of VDAC1oligomerization is a piperidine derivative having the chemical formula of (1-(naphthalen-2-ylmethyl)-4-(phenylamino) piperidine-4-carbonyl)glycine, designated as VBIT-12, or an enantiomer, a diastereomer, or a salt thereof.

53. The method of claim 42, wherein the at least one VDAC modulator is a protein, a peptide or an antibody.

54. The method of claim 42, wherein the at least one VDAC modulator is a small molecule drug or agent, or a small RNA molecule interfering with the expression and/or activity of the at least one VDAC.

55. The method of claim 49, wherein the modulator of VDAC1 is a modulator interfering with the VDAC1 pro-apoptotic activity.

56. The method of claim 42, wherein said slowing the progression or treating the skin disorder associated with a defect of epidermal and/or subepidermal cells adhesion comprises a reduction of apoptosis in the epidermal and/or subepidermal tissue samples of said subject.

57. The method of claim 42, wherein said slowing the progression or treating the skin disorder associated with a defect of epidermal and/or subepidermal cells adhesion comprises a reduction of acantholysis in the epidermal and/or subepidermal tissue samples of said subject.

58. The method of claim 42, wherein said slowing the progression or treating the skin disorder associated with a defect of epidermal and/or subepidermal cells adhesion comprises at least one of:
a. a reduction of activity of at least one of Caspase 3, 7 and/or 9 in the epidermal and/or subepidermal tissue samples of said subject,
b. a reduction of transcription activity of p53 in the epidermal and/or subepidermal tissue samples of said subject,
c. an increase in the expression of Bcl-2 gene or protein in the epidermal and/or subepidermal tissue samples of said subject.

59. The method of claim 42, wherein the at least one VDAC modulator is formulated for sustained, controlled or targeted release of the modulator into epidermal, subepidermal or mucosal tissue.

60. The method of claim 42, further comprising administering to said subject at least one additional therapeutic agent, either in the same formulation with or independently of the at least one VDAC modulator.

61. An oral or a topical composition for slowing the progression or treating a skin disorder associated with a defect of epidermal and/or subepidermal cells adhesion, the composition comprising an effective amount of at least one modulator of at least one voltage-dependent anion channel isoform VDAC1, 2 or 3.
